(19) United States
(12) Patent Application Publication
(10) Pub. No.: US 2019/0233072 A1
(43) Pub. Date: Aug. 1, 2019

(54) WATER PROPULSION APPARATUS

(71) Applicants: Richard William Rose, Brecksville, OH (US); Alex Greenspan, Solon, OH (US)

(72) Inventors: Richard William Rose, Brecksville, OH (US); Alex Greenspan, Solon, OH (US)

(21) Appl. No.: 16/261,272

(22) Filed: Jan. 29, 2019

Related U.S. Application Data
(60) Provisional application No. 62/709,753, filed on Jan. 29, 2018.

(51) Int. Cl. B63H 1/16; A61B 5/0205; A61B 5/01; A61B 5/0402; A61B 5/00

(52) U.S. Cl. CPC B63H 1/16; A61B 5/02055; G01S 19/42; A61B 5/0402; A61B 5/6895; A61B 5/01

(57) ABSTRACT

The present disclosure relates to a water propulsion apparatus. In some examples, a water propulsion apparatus comprises a body extending from a proximal end to a distal end. A handle is provided on the proximal end. A blade is provided on the distal end and has an opening extending entirely through the blade. An impeller is positioned within the opening and is rotatable to provide automated thrust to the apparatus.

### CROSS-REFERENCE TO RELATED APPLICATION

This application claims the benefit of priority from U.S. Provisional Patent Application No. 62/709,753, filed 29 Jan. 2018, and entitled “SMART OAR,” which is incorporated herein by reference in its entirety.

### TECHNICAL FIELD

The present disclosure relates to water propulsion. More particularly, the present disclosure relates to a water propulsion apparatus.

### BACKGROUND

Oars, paddles and like water propulsion devices usually include a shaft having an integral blade at one end thereof. When the device is moved through water, the device causes a reaction which propels a water vehicle (e.g., a ship, a boat, a kayak, etc.). At the end of each stroke or movement of the device, it is lifted out of the water, returned to its initial position and the propelling stroke is repeated.

### SUMMARY

In an example, a water propulsion apparatus comprises a body extending from a proximal end to a distal end. A handle is provided on the proximal end. A blade is provided on the distal end and has an opening extending entirely through the blade. An impeller is positioned within the opening and is rotatable to provide automated thrust to the apparatus.

In another example, a water propulsion apparatus comprises a body extending from a proximal end to a distal end. A handle is provided on the proximal end. A blade is provided on the distal end and has an opening extending entirely through the blade. A motor is connected to the blade. A cover is connected to the body and movable relative to the blade from a closed condition covering the opening to an open condition spaced entirely from the opening in the blade. An impeller positioned within the opening and rotatably connected to the motor to provide automated thrust to the apparatus when the cover is in the open position.

### BRIEF DESCRIPTION OF THE DRAWINGS

FIG. 1A is a perspective view of an example water propulsion apparatus.

FIG. 1B illustrates a side-view of a portion of the water propulsion apparatus of FIG. 1A.

FIG. 2 illustrates an example control device of the water propulsion apparatus.

FIG. 3 illustrates an example housing for the control device of FIG. 2.

FIG. 4 illustrates a cover of the water propulsion apparatus in a closed condition.

FIG. 5 illustrates the cover of the water propulsion apparatus in an open condition.

### DETAILED DESCRIPTION

The present disclosure relates to water propulsion. More particularly, the present disclosure relates to a water propulsion apparatus.

The water propulsion apparatus can be employed to provide water-borne propulsion for a user or a water vehicle, either manually or in an automated manner. For example, the user can hold the water propulsion apparatus with two hands, some distance apart from each other, and pass a blade of the water propulsion apparatus through the water to manually generate thrust to move the water vehicle in a given direction. An impeller on the blade is covered by a sliding cover during manual thrust generation.

The impeller can be coupled directly to a shaft of the motor on the water propulsion apparatus (i.e., no gear reduction is required to couple the impeller to the motor). The cover can be slid away from the impeller to expose the impeller such that actuating the motor causes rotation of the impeller. This generates thrust in an automated manner to move the water vehicle or the user through the water in the given direction.

In some examples, the water propulsion apparatus can be substantially fabricated from carbon fiber such that the water propulsion is light weight, durable and can float on the water. To this end, the user can employ the water propulsion apparatus when not in the water vehicle to provide buoyancy. Accordingly, the water propulsion apparatus can function as a lifebuoy to propel the user through the water (e.g., to safety, another location, etc.). For example, during an emergency (e.g., after or during capsizing) the user can fall into the water. While in the water, the user can grasp the water propulsion apparatus and actuate the motor to generate thrust and move the user through the water to safety.

The water propulsion apparatus described herein can be utilized in various applications including nautical sports, navigation, water emergencies, recreational events, law enforcement applications, military applications (e.g., training), as a safety element (e.g., a floating device), reconnaissance applications (e.g., provide reconnaissance of distressed water vehicles), etc. Although the water propulsion apparatus is described herein in relation to an oar, the examples described herein should not be construed and/or limited to only oars, and can include paddles, or similar water propulsion devices.

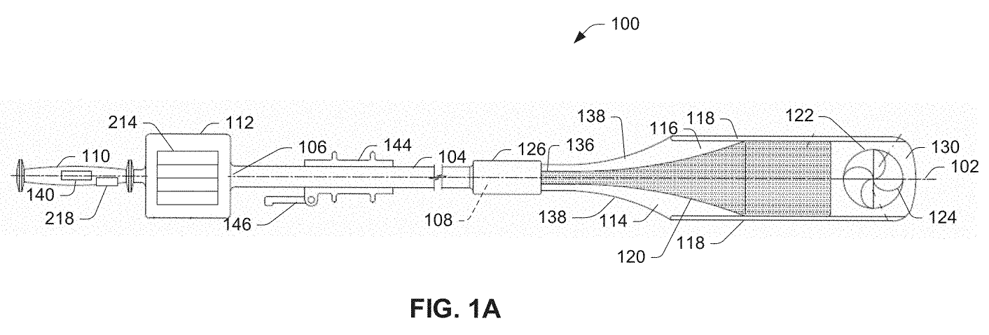

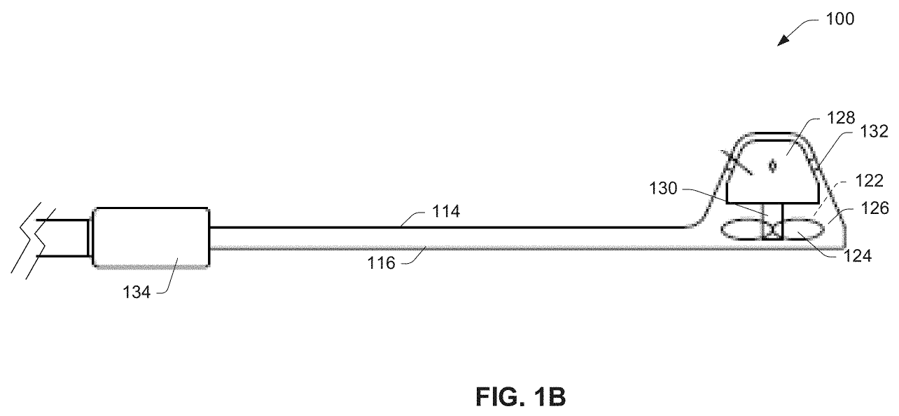

FIGS. 1A-1B illustrate an example water propulsion apparatus 100 for helping propel a user or a water vehicle (e.g., a boat, kayak raft, etc.) through water. The water propulsion apparatus 100 extends longitudinally along an axis 102 and includes a body 104. The body 104 extends from a proximal end 106 to a distal end 108. A handle 110 and a control device 112 are connected to the distal end 108. In an example, the handle 110 is configured to float and can therefore be used by the user as a floating beacon.

A blade 114 is provided at the distal end 108 and includes a blade face 116. Curved edges 118 extend from the blade face 116 towards one another and towards the axis 102 to define curved grooves for slidably receiving a cover 120. While holding the handle 110 with one hand and the body 104 with the other hand, the user can draw the apparatus 100 through the water in any direction (e.g., from front to back or back to the front) to manually generate thrust to move the water vehicle in a given direction (e.g., in a forward direction or a reverse direction).

An opening 122 extends entirely through the blade 114 and receives an impeller 124. More specifically, the opening 122 can extend from the blade face 116 of the blade 114 through a thickness of the blade 114 perpendicular to the axis 102. The depth of the opening 122 can be a function of the blade thickness. In some examples, the opening 122 can be constructed to gradually decrease in diameter in a direction extending through the blade 114 and away from the blade face 116.

A motor housing 126 is formed integrally with the blade 114 or connected thereto. In either case, the motor housing 126 is aligned with the opening 122. The motor housing 126 can include openings 132 (e.g., passageways) in fluid communication with the opening 122 to pass through the water drawn into the opening 122 by the impeller 124 in order to generate the thrust. A motor 128 can be secured to inner sidewalls of the motor housing 126 by a coupling mechanism (e.g., screws). The motor 128 includes a shaft 130 extending into the opening 122 and coupled directly to the impeller 124. As such, the impeller 124 can be coupled directly to the shaft 130 without reduction gearing. In some examples, the motor 128 can correspond to a direct-current (DC) motor (e.g., a 48 V DC motor). As described herein, the motor 128 provides power to the impeller 124 to turn the impeller 124 and generate thrust for moving the water vehicle or the user across the water. The impeller 124 can be a multi-stage impeller (e.g., a two-stage impeller, three-stage impeller, etc.) and/or turbo jet impeller and can be constructed from a carbon fiber material. The multi-stage impeller can create a hydro jet propulsion to thrust the water vehicle or the user in a given direction.

The cover 120 has an open condition spaced from the opening 122 and a closed condition covering the opening. In other words, the cover 120 is movable along the axis 102 to selectively cover the opening 122. The cover 120 is placed in the closed condition while the user is manually drawing the blade 114 through the water to generate thrust and to move the water vehicle in the given direction. The cover 120 is placed in the open condition when it is desirable to operate the impeller 124 to propel the water vehicle or the user through the water in an automated manner.

A bushing 134 extends over the body 104 and a flared or tapered end 136 of the blade 114 and is longitudinally movable relative thereto. The bushing 134 is secured to the cover 120 such that longitudinal movement of the bushing 134 causes the cover 120 to move relative to the blade 114 and, thus, move relative to the opening 122. As a result, the user can move the bushing 134 towards the opening 122 until the cover 120 reaches the closed condition covering the opening 122.

The user can likewise move the bushing 134 towards the handle 110 until the cover 120 is placed in the open condition spaced entirely from the opening 122. Portions 138 of the blade 114 can be flared or tapered to form an abutment for the bushing 134 that provides tactile feedback to the user when the cover 120 has reached the closed condition. The bushing 134 can be formed from a resilient material that expands when the bushing 134 reaches the portions 138 of the blade 114 to form a friction fit that automatically locks the bushing 134 in place when the cover 120 reaches the closed condition.

The control device 112, in response to receiving the signals from the touch sensor 142, actuates the motor to turn the impeller 124 and generate thrust to propel the user or the water vehicle in an automated manner. This configuration helps to ensure that the impeller 124 is only driven when the user contacts the touch sensor 136 a period of time indicative of wishing to propel the user or the water vehicle in an automated manner (e.g., quickly touching and releasing the touch sensor will not cause the impeller 124 to rotate).

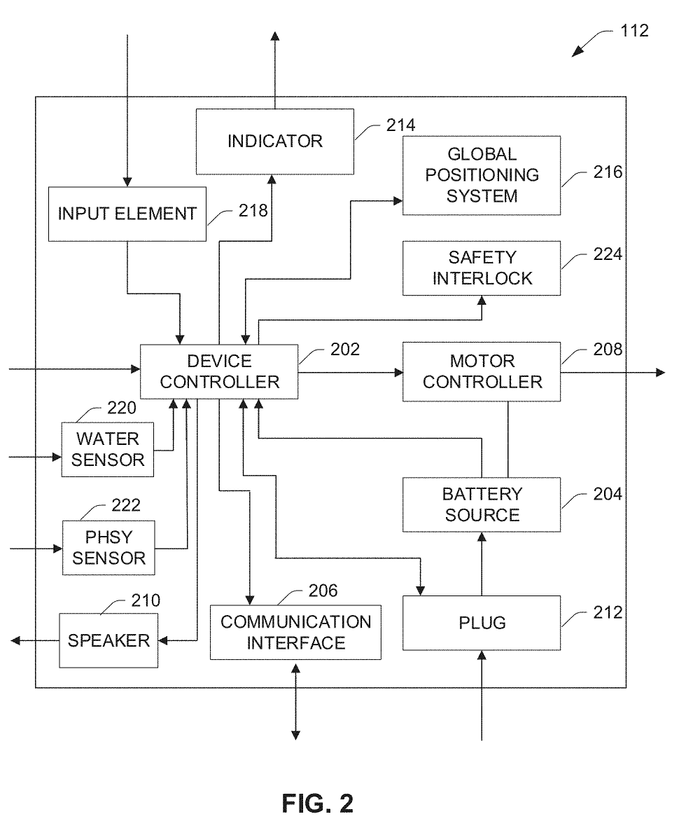

An example configuration of the control device 112 is shown in FIG. 2. The control device 112 can include a device controller 202, a battery source 204, a communication interface 206, a motor controller 208, a speaker 210, an electrical plug 212, one or more indicators 214, a global positioning system (GPS) 216, an input element 218, a water sensor 220, one or more physiological sensors 222, and a safety interlock 224.

The battery source 204 can include one or more batteries (e.g., rechargeable batteries, such as lithium-ion batteries, or deep-cycle batteries). The battery source 204 can be removed from the apparatus 100 for external charging or fixed to the apparatus and charged via the electrical plug 212 when connected to an external power source (not shown). The controller 202 is connected to the battery source 204 and the electrical plug 212 and can cease the flow of electricity from the electrical plug to the battery source when the battery source is sufficiently charged.

The controller 202 is connected to the indicator 214 and actuates the same to indicate different conditions of the battery source 204 (e.g., the power level, when the power level is below a threshold amount, when the battery source needs charging, etc.). The indicator 214 can also indicate when the electrical plug 212 is connected to the external power source and/or when the battery source 204 is being charged by the external power source. To this end, the indicator 214 can include one or more light sources (e.g., one or more light emitting diodes (LEDs)) that indicate different conditions of the battery source 204 in different manners (e.g., by emitting different colors, flashing, etc.).

The indicator 214 can also act as an emergency beacon initiated automatically to transmit based on sensed conditions or by the user to output a beacon at a given radio-frequency (RF) that can be identified by an external source (e.g., RF receiver) or one or more lights can also be activated. In other words, the indicator 214 can generate and send RF signals, as well as visual indicators, to identify an emergency to persons other than the user. As such, if the user is in distress or in need of immediate rescue, the emergency beacon can be initiated so that appropriate personnel (e.g., rescue personnel) can assist the user.

In an example, the water sensor 220 sends a signal to the controller 202 in response to coming in contact with water or being submerged under water for a given period of time (e.g., five seconds). The controller 202, in response to receiving the signal, can direct the indicator 214 to initiate the emergency beacon. As such, the controller 202 can be configured to cause the indicator 214 to emit or display the beacon based on the signal from the water sensor 220.

The GPS 216 is connected to the controller 202 and is configured to communicate with one or more satellites (not shown) to determine location information for the water propulsion apparatus 100. The controller 202 can be configured to cause the GPS 216 (e.g., periodically) to determine the location information for the water propulsion apparatus 100. In response thereto, the controller 202 can cooperate with the indicator 214 to indicate (e.g., via different colored LEDs) to the user when the location has been obtained and/or when a satellite connection has been made.

The location data collected by the GPS 216 and received by the controller 202 can be stored in memory on board the control device 112 and/or sent to an external device (not shown) via the communication interface 206. The external device can include one or more of a laptop computer, a desktop computer, a tablet computer, a workstation, or the like. The communication interface 206 can be configured to communicate wirelessly (e.g., using Bluetooth, WiFi, etc.) and/or over a wired connection (e.g., a physical cable) with the external device.

In some examples, the communication interface 206 can include a universal serial bus for communicating data to the external device. The external device can be configured to execute a special-purpose application to receive and display the location information on a display of the external device. In some examples, the control device 112 can include a display (not shown) for displaying the location information.

The motor controller 208 is connected to the impeller 124 (FIG. 1A) and the controller 202 for controlling the speed and/or direction of rotation of the impeller 124. Rotation of the impeller 124 can be controlled in response to the user or the external device. With this in mind, the communication interface 206 can be employed to provide and/or receive data from the external device. The data can include impeller data and can be used by the controller 202 to control the motor controller 208 to regulate the speed and/or direction of the impeller 124. The motor controller 208 can also be configured to provide a motor current to the motor 128 based on the charge stored at the battery source 204. As such, the motor controller 208 can be configured to drive the motor 128 based on the charge stored at the battery source 204.

In regards to the user, the input element 218 can include one or more buttons or combinations thereof (represented schematically in FIG. 1A) that send signals to the controller 202 for controlling rotation of the impeller 124 in response to user interaction with the input element. The input element 218 can constitute a touch sensor (e.g., a capacitive or optical touch sensor) provided on the handle 110. To this end, the input element 218 sends signals to the control device 112 when the user touches the input element 218 for greater than a predetermined amount of time. In other words, the input element 218 can be configured to distinguish grabs and touches. Touches can be defined as being less than a given amount of time (e.g., less than about 300 milliseconds (ms)), and grabs can be defined as at or more than the given amount of time (e.g., 300 ms or longer). This configuration helps to ensure the impeller 124 is only driven when the user contacts the input element 218 a period of time indicative of wishing to propel the user or the water vehicle in an automated manner (e.g., quickly touching and releasing the input device will not cause the impeller 124 to rotate).

In one example, the input element 218 can include a forward input, an off input, and/or a reverse input that each generate a signal indicative of a user input. In response to receiving a forward input signal, the controller 202 causes the impeller 124 to rotate in a clockwise direction to propel the water vehicle or the user in the water in a first given direction (e.g., a forward direction). In response to receiving a reverse input signal, the controller 202 causes the impeller 124 to rotate in a counter-clockwise direction to propel the water vehicle or the user in the water in another given direction (e.g., a reverse direction). In response to receiving an off input signal, the controller 202 stops rotation of the impeller 124 to thereby cease propulsion of the water vehicle or user in any particular direction. The controller 202 can be configured to generate an audible signal indicative of the input signal received and actuate the speaker 210 to emit the audible signal to the user.

The speed of the impeller 124 can be controlled by the user via a potentiometer. The potentiometer can corresponding to an input element (e.g., the input element 218)). The speed of the impeller 124 can be increased by turning the potentiometer in a given direction (e.g., clock-wise direction), while turning the potentiometer in another direction (e.g., counter clock-wise direction) decreases the speed of the impeller 124.

The one or more physiological sensors 222 can be connected to the user (e.g., via leads) and/or integrated into the handle 110 for monitoring a physical condition of the user while the user is utilizing (e.g., gripping) the water propulsion apparatus 100. The physiological sensors 222 can include an electrocardiogram (ECG or EKG) sensor, a blood pressure sensor, a body temperature sensor, etc. As such, physiological conditions of the user can be monitored while the user is utilizing the water propulsion apparatus 100.

The controller 202 can be configured to communicate the physiological condition data to the external device via the communication interface 206. The external device can be configured to display on the display the physiological condition data. In an example, the controller 202 can be configured to evaluate the physiological condition data relative to baseline physiological condition data (e.g., data for a healthy user or a group of users) stored in memory to determine the user's physical condition. In response to determining that the user's condition has deviated from the baseline physiological condition data, the controller 202 can be configured to cause the indicator 214 to emit an indication for the user (e.g., a blinking light). Additionally, or alternatively, the controller 202 can be configured to cause the emergency beacon to emit the beacon at the given RF in response to determining that the user's condition has deviated from the baseline physiological condition data.

Referring to FIG. 1A, the handle 110 can include an emergency release pressure sensor 140 connected to the handle or integrally formed therewith. The emergency release pressure sensor 140 can be actuated by the user (e.g., by pressing against the sensor 140) to cut power to the motor 128. Additionally, or alternatively, the emergency release pressure sensor 140 can be actuated by the user to cause one or more indicators (e.g., the indicator 214) to emit one or more lights (e.g., signal lights, such as distress lights).

Referring back to FIG. 1A, the body 104 can be formed from a series of telescoping tubes (not shown) that allow the length of the apparatus 100 to be varied along the axis 102. A locking mechanism 144 is provided on the body 104 and includes a lock 146 (e.g., a pivoting handle). The lock 146 can be disengaged from the body 104 by the user to allow the length of the apparatus 100 to be adjusted by telescoping action. The lock 146 can be engaged with the body 104 by the user to fix the length of the apparatus 100. For example, the lock 146, when engaged, can create a force perpendicular to the axis 102 that prevents telescoping movement of the apparatus 100. In one example, the locking mechanism 144 is a u-joint locking mechanism and the lock 146 is a lock lever. Portions of the water propulsion apparatus 100, including the handle 110, the body 104, the blade 114, and the locking mechanism 144 can be hollow and/or constructed from a carbon fiber material. The safety interlock 224 can be coupled to the locking mechanism 144 and can be configured to cooperate with the controller to prevent the impeller 124 from being rotate so long as the cover 120 is in the closed position covering the impeller 124.

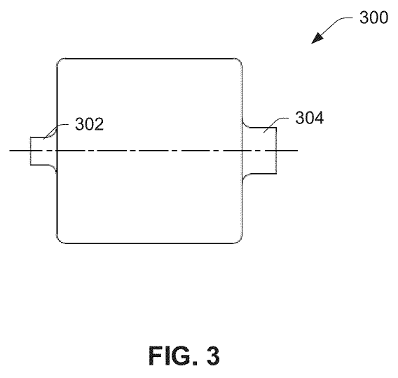

FIG. 3 illustrates an example of a control device housing 300 for the control device 112. A tubular, first hollow body portion 302 of the control device housing 300 receives the handle 110. A tubular, second hollow body portion 304 receives the proximal end 106 of the body 104. The housing 300 can be waterproof and constructed from a lightweight, durable material, (e.g., carbon fiber). One or more elements of the control device 112 are sealed within the housing 300 to protect the elements from the surrounding environment. As such, the housing 300 can provide an air-tight seal around one or more elements of the control device 112. Certain features of the control device 112, however, such as the water sensor 220 can protrude from or be exposed through the housing 300 (not shown in FIG. 3) for water sensing, as described herein.

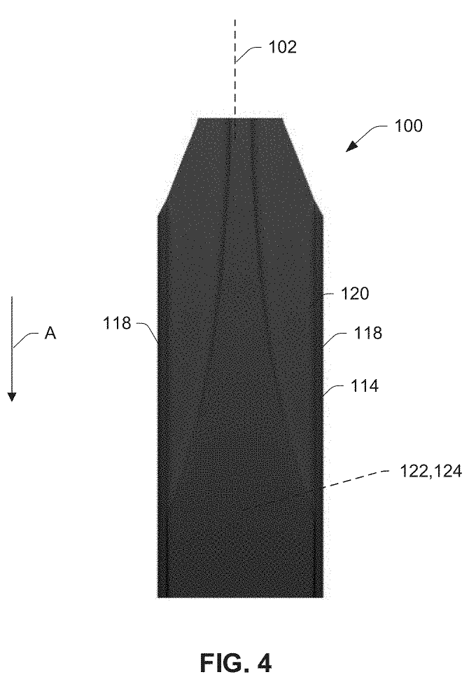

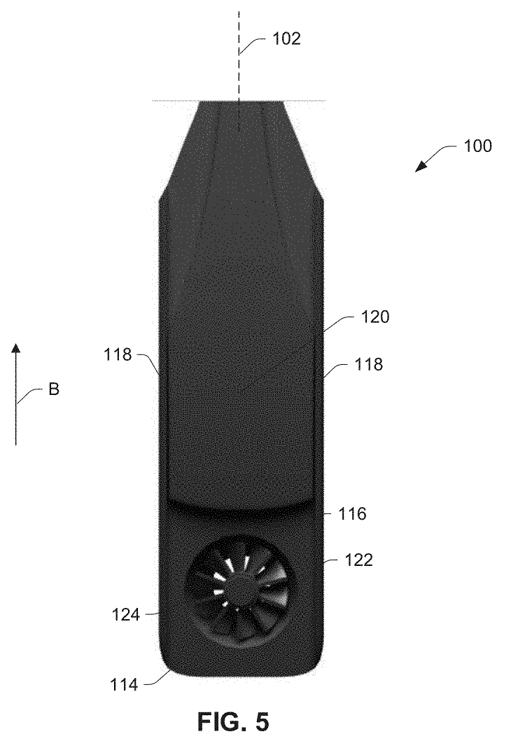

Referring to FIG. 4, for manual operation of the water propulsion apparatus 100, the cover 120 is moved in the direction A along the axis 102 to the closed condition covering the opening 122 and thereby preventing the impeller 124 from providing thrust. Referring to FIG. 5, when automated thrust is needed/desired, the cover 120 is moved in the direction B to the open condition spaced entirely from the opening 122 and impeller 124. This allows the impeller 124 to be controlled by the user to provide an automated thrust to the water propulsion apparatus 100. To this end, when the cover 120 is in the open condition, operating the input element 218 provides the corresponding signal (e.g., the forward signal, the reverse signal, etc.) to the controller 202 which, in turn, directs the motor controller 208 to operate the impeller 124. Accordingly, while the cover 120 is in the open position, the impeller 124 can generate thrust to move the water vehicle or the user across the water in a desired manner. The controller 202 can prevent rotation of the impeller 124 when the cover 120 is in the closed position.

In some examples, the water propulsion apparatus 100 can be used in emergency applications. For example, while moving a water vehicle across water, the water vehicle may be intentionally or unintentionally overturned, or may experience dangerous waters and/or weather conditions that may result in the water vehicle being overturned, thereby forcing the user into the water. In response, the user can operate the water propulsion apparatus 100 to help reach a safer location. For example, the user, while in the water, can operate the input element 218 to cause the impeller 124 to generate thrust while the user holds the water propulsion apparatus 100. This propels the user through the water to help the user get to safety. Accordingly, the water propulsion apparatus 100 can allow the user to get to safety quickly and effectively.

In some examples, the water propulsion apparatus 100 can be used to provide thrust while the user is within the water vehicle. For example, the user can partially position the blade 114 in the water while the cover 120 is in the open condition such that the opening 122 and therefore the impeller 124 are submerged under water. The user can then operate the input element 218 to cause rotation of the impeller 124 to generate thrust by drawing water with the rotating impeller 124 into the opening 122 and discharging the water out of the openings 132 in the motor housing 126. The openings 132 of the motor housing 126 can therefore be configured to direct the water in a desired direction to provide thrust. The rotation of the impeller 124 and orientation of the blade 114 in the water relative to the water vehicle will dictate the direction the thrust is imparted and therefore dictate the direction the water vehicle moves in response thereto.

Accordingly, the water propulsion apparatus 100 can create thrust for moving the water vehicle in an automated manner without requiring the user to manually generate the thrust to move the water vehicle across the water. The water propulsion apparatus 100 can be used by water vehicle operators that have limited or no knowledge of capsize processes (e.g., responding and/or recovering from a partial or complete capsize). In some examples, the water propulsion apparatus 100 can permit the user to conserve energy in emergency situations and not expend a substantial amount of physical energy in implementing capsize procedures. For instance, if the water vehicle is overturned, the user can utilize the water propulsion apparatus 100 to propel the user to an intended location (e.g., a safe location, land, another boat, etc.) without exerting substantial amount of physical energy to implement capsize procedures.

It will be appreciated, however, that the water propulsion apparatus 100 is configured to allow the user to manually provide thrust to the water vehicle in a conventional manner by paddling, which is done with the cover 120 in the closed position.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

What is claimed is:

1. A water propulsion apparatus comprising:
a body extending from a proximal end to a distal end;
a handle provided on the proximal end;
a blade provided on the distal end and having an opening extending entirely through the blade; and
an impeller positioned within the opening and being rotatable to provide automated thrust to the apparatus.

2. The water propulsion apparatus of claim 1 further comprising a cover connected to the body and movable relative to the blade from a closed condition covering the opening to an open condition spaced entirely from the opening in the blade

3. The water propulsion apparatus of claim 2, further comprising a bushing coupled to the cover and configured to move the cover longitudinally from the open condition to the closed condition relative to the blade.

4. The water propulsion apparatus of claim 2, wherein the blade further comprises curved edges extending inwardly toward one another to define grooves for slidably receiving the cover.

5. The water propulsion apparatus of claim 1, wherein the impeller is a multi-stage impeller.

6. The water propulsion apparatus of claim 2, wherein the blade and the impeller are constructed from a carbon fiber material.

7. The water propulsion apparatus of claim 2, further comprising a motor coupled to the blade, wherein the impeller is coupled directly to a shaft of the motor without a gear reduction mechanism.

8. The water propulsion apparatus of claim 1, wherein the opening gradually decreases in diameter through the blade.

9. The water propulsion apparatus of claim 1, further comprising:
a motor for driving the impeller; and
and an emergency release pressure sensor provided on the handle and configured to cut power to the motor to cease providing the automated thrust to the apparatus.

10. The water propulsion apparatus of claim 1, further comprising a control device connected to the body and being configured to control at least one of a speed and a rotational direction of the impeller.

11. The water propulsion apparatus of claim 10, further comprising a touch sensor configured to provide a signal indicative of user touch of the handle, wherein the control device controls at least one of the speed and the rotational direction of the impeller based on the signal.

12. The water propulsion apparatus of claim 10, further comprising:
a water sensor configured to provide a signal when the apparatus comes into contact with the water or is submerged under water for a given period of time; and
an indicator configured to emit an indication based on the signal.

13. The water propulsion apparatus of claim 12, wherein the control device further comprises:
a global position system (GPS) configured to determine location information for the apparatus; and
a GPS indicator configured to emit an indication in response to determining the location information.

14. The water propulsion apparatus of claim 10, further comprising one or more physiological sensors configured to monitor physiological conditions of a user of the apparatus, wherein the one or more physiological sensors comprises one of an electrocardiogram sensor, a blood pressure sensor, and a body temperature sensor.

15. The water propulsion apparatus of claim 1, further comprising a control device connected to the body, the control device comprising a controller and an input element, wherein the input element is configured to generate an input signal, the controller being configured to control one of a rotation and speed of the impeller based on the input signal.

16. A water propulsion apparatus comprising:
a body extending from a proximal end to a distal end;
a handle provided on the proximal end;
a blade provided on the distal end and having an opening extending entirely through the blade;
a motor connected to the blade;
a cover connected to the body and movable relative to the blade from a closed condition covering the opening to an open condition spaced entirely from the opening in the blade;
an impeller positioned within the opening and rotatably connected to the motor to provide automated thrust to the apparatus when the cover is in the open position.

17. The water propulsion apparatus of claim 16, wherein the impeller is a multi-stage impeller.

18. The water propulsion apparatus of claim 17, wherein the impeller is coupled directly to the shaft without a gear reduction mechanism.

19. The water propulsion apparatus of claim 18, further comprising a bushel coupled to the cover and configured to move the cover longitudinally from the open condition to the closed condition relative to the blade.

20. The water propulsion apparatus of claim 19, wherein the blade further comprises curved edges extending inwardly toward one another to define grooves for slidably receiving the cover.
